(19) United States
(12) Patent Application Publication
(10) Pub. No.: US 2003/0180347 A1
(43) Pub. Date: Sep. 25, 2003

(54) Patch for the delivery of topical agents

(75) Inventors: Tyler F. Young, Hampden, MA (US); Harry Hayes, Hampden, MA (US)

Correspondence Address: Kevin H. Vanderleeden, McCormick, Paulding & Huber LLP, 185 Asylum Street, CityPlace II, Hartford, CT, 06103, US

(73) Assignee: W.F. YOUNG, INCORPORATED, East Longmeadow, MA (US)

(21) Appl. No.: 10/388,300

(22) Filed: Mar. 13, 2003

Related U.S. Application Data
(60) Provisional application No. 60/365,193, filed on Mar. 19, 2002.

(51) Int. Cl. A61K 9/70

(52) U.S. Cl. 424/449

(57) ABSTRACT

The adhesive patch for the delivery of topical agents to the skin including a polymer matrix, generally a hydrogel matrix having adhesive properties, a skin conditioner and a penetration enhancer. The hydrogel matrix including purified water, glycerin, polyacylate, sorbitol, kaolin, CMC (carboxymethyl cellulose), alcohol, castor oil, TWEEN 80 (polyoxyethylene sorbitan monooleate), fragrance and citric acid. Skin conditioners and penetration enhancers including methyl sulfonyl methane, glucosamine and chondroitin. The matrix may also include a topically effective drug. The adhesive patch includes a support backing of non-woven material or a non-occlusive film and a protective film covering the polymer matrix.

### CROSS REFERENCE TO RELATED APPLICATION

This application claims the benefit of U.S. Provisional Application Serial No. 60/365,193, filed on Mar. 19, 2002.

### FIELD OF THE INVENTION

The present invention relates to a patch for the delivery of topical agents to the skin. In particular, the present invention involves a patch that utilizes a polymer matrix containing an adhesive, a skin conditioner, a penetration enhancer, and, optionally, a topically effective drug. The polymer matrix facilitates the slow diffusion of the topical agents' active ingredients over a period of time. Additionally, the polymer matrix provides adhesion such that the topical agents are kept in contact with the skin for a period of time sufficient for the agents to be effective.

### BACKGROUND OF THE INVENTION

The delivery of topical agents to the skin may be desirable to improve the health and appearance of skin and alleviate conditions such as psoriasis, dry skin, blemishes, abrasions, cuts, or rashes. If such skin conditions are not treated, further damage may occur leading to infections and other maladies. Additionally, the treatment of chronic or acute pain often involves the topical application of an anesthetic such as lidocaine or benzocaine.

Generally, topically applied treatments are transient. They are applied by rubbing an agent into the skin. Topically applied treatments are usually in the form of creams, lotions or gels that are applied and dispersed by rubbing the mixture until it is absorbed by the skin. To ensure that there is no residue left on the skin, the mixtures typically contain a volatile component to evaporate from the skin's surface, a surfactant to ensure breakdown of the surface tension of the skin to allow penetration as well as active ingredients to treat the particular condition.

For many conditions, however, optimal treatment of the condition only occurs after the topical agent has been in contact with the skin for a longer period of time than can be obtained by rubbing a cream, lotion or gel into the skin. For example, non-steroidal anti-inflammatory agents such as ketoprofin obtain optimal permeation of the skin only after being in contact with skin for 15-20 hours.

Further, it may be desirable to prevent the wearer of the patch from scratching or otherwise touching the area being treated.

Accordingly, there is a need for a patch that allows a topical agent, such as a skin conditioner, to treat a skin condition over a period of time that cannot be obtained through the use of a transient topical treatment.

It is an object of the present invention to provide a patch for the topical treatment of skin conditions.

It is a further object of the present invention to provide a patch for the topical application of a skin conditioner for the treatment of dry skin, psoriasis and other skin conditions.

It is a further object of the present invention to provide a patch for the topical application of drugs such as lidocaine and topical analgesics such as menthol, lidocaine, methyl salicylate or trolamine salicylate.

It is a further object of the present invention to provide a patch that allows a topical agent to remain in contact with skin for a period of time.

It is a further object of the present invention provide a patch that prevents the wearer of the patch from scratching or otherwise touching the area being treated.

### SUMMARY OF THE INVENTION

A patch made in accordance with the present invention provides a polymer matrix for the delivery of topical agents, such as skin conditioners, to the skin. In a preferred embodiment, the patch includes a polymer matrix, either a hydrogel matrix or oil-based latex gel matrix, that includes an adhesive, a skin conditioner and a penetration enhancer. Optionally, the matrix may include a drug ingredient such as lidocaine, menthol, lidocaine or methyl salicylate. In a preferred embodiment, the polymer matrix is contained on a backing that provides structural support for the matrix. Preferably, the backing is manufactured from a non-woven material or a non-occulsive film. Additionally, in a preferred embodiment, the matrix is covered by a protective sheet that is removed just prior to its application.

The polymer matrix of the present invention facilitates the slow diffusion of the topical agents' active ingredients over a period of time. Additionally, the polymer matrix provides adhesion such that the topical agent is kept in contact with the skin for a period of time sufficient for the agent to be effective.

The present invention completely covers the treatment area such that the wearer cannot easily access the area.

### BRIEF DESCRIPTION OF THE DRAWINGS

FIG. 1 is a perspective view of the back of a hand depicting the patch of the present invention applied to cover a skin condition.

FIG. 2 is a top perspective view of the patch of FIG. 1 showing the backing, polymer matrix and protective film.

FIG. 3 is a cross-sectional view on an enlarged scale of the patch of FIGS. 1 and 2 depicting the patch as it appears when applied to the patient's skin.

FIG. 4 is a graph showing the effect over time of a hydrating patch on water concentration in keratin.

FIG. 5 is a graph showing skin permeation of Ketoprofen over a period of time.

### DETAILED DESCRIPTION OF THE PREFERRED EMBODIMENT

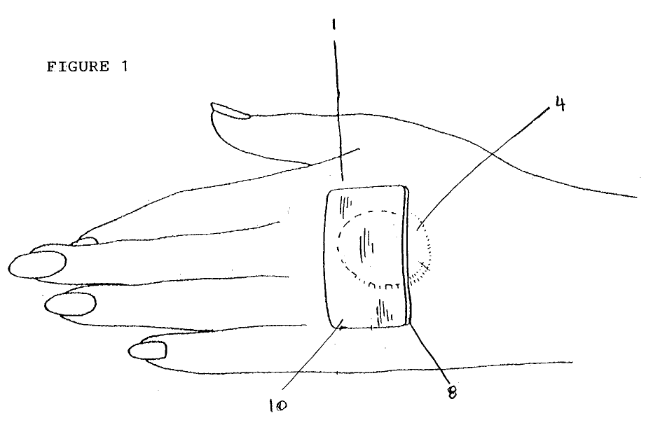

Referring to FIG. 1, the patch 1 of the present invention has been applied to a patient's hand to cover and treat a skin condition 4 such as psoriasis. The patch, which is rectangular, preferably has a water-based hydrogel matrix layer 8 and a backing layer 10 that provides structural support for the patch. The matrix may also be an oil-based latex gel.

The backing layer 10 is preferably either a non-woven material or a non-occlusive film. The non-woven material may be either a natural material such as from the cotton, flax, linen, paper or cellulose families, a naturally-derived material such as viscose rayon or solvent spun rayon, or a synthetic material such as those from the polyamide, polyester, polyolefin, polyurethane and polyacrylate families. The non-woven material may also be a mixture or two or more of the above mentioned materials. The non-occlusive film is preferably comprised of polypropylene, polyethylene, polyurethane or mixtures thereof. In its preferred configuration, the film is either a low-density polyethylene or polyurethane film. Regardless of the material, the backing layer should be nonirritating to skin.

A suitable backing layer is a nonwoven fabric comprising a wetlay cellulose and polyester nonwoven fabric containing as a sizing an acrylic latex emulsion resin available from Dexter Corporation of Windsor Locks, Conn.

The backing layer may also include a slippery friction-reducing layer such as a sheet of polyethylene. The friction-reducing layer would function to promote sliding contact and eliminate chafing between the patch 1 and an article of clothing placed over the patch.

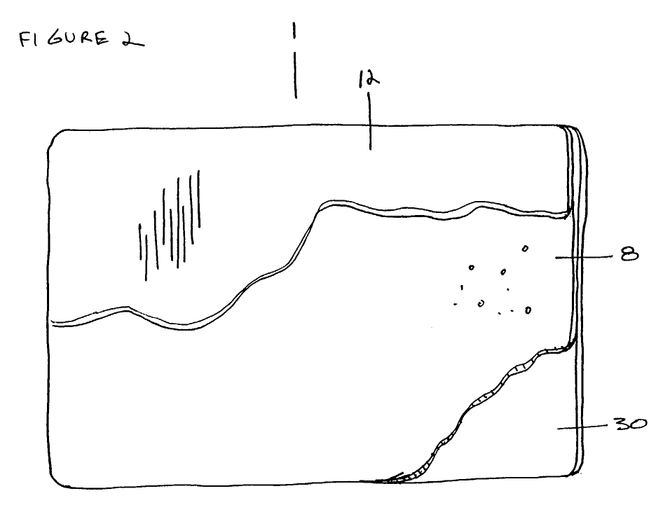

Now referring to FIG. 2, the patch 1 also includes a protective sheet 12 which removably covers the polymer matrix 8. The protective sheet covers the matrix prior to the application of the patch and protects it during shipment and storage. The protective sheet may be a slip sheet or liner sheet of any suitable commercially available composition. For example, the protective sheet 12 may be a 2 mil. sheet of polyester film.

The patch is typically about three inches long by four inches wide and has rounded corners. It may also be circular. However, other sizes are possible depending upon the area of skin to be covered.

The polymer matrix usually has several ingredients. The ingredients include a liquid phase of the matrix, which preferably consists of hydric alcohols such as glycerol and/or propylene glycol, and/or water. The matrix also contains a hydrophilic polymer, which acts as a thickening agent. The polymer may be either natural such as starch, kelp, sorbitol or gum or synthetic such as CMC (carboxymethyl cellulose). In its preferred configuration, the base ingredients of the matrix include in decreasing order of concentration: purified water, glycerin, polyacylate, sorbitol, kaolin, CMC (carboxylmethyl cellulose), alcohol, castor oil, TWEEN 80 (polyoxyethylene sorbitan monooleate), fragrance and citric acid.

Examples of the percentage ranges of the ingredients in an embodiment of the present invention are as follows:

| Ingredient: | % Range: |
|---|---|
| L-Menthol | 0.1-1 |
| Menthol Oil | 0.9-15 |
| N-acetyl glucosamine | .0001-2 |
| Methyl sulfonyl methane | .00005-2 |
| Alcohol | .5-10 |
| TWEEN 80 | .5-2 |
| Water | 0-90 |
| Glycerin | 0-25 |
| Sorbitol | 0-30 |
| Polyacrylates | 10-35 |
| CMC | .05-6 |
| Citric Acid | .01-.6 |
| Latex | 0-90 |

The polymer matrix 8 has adhesive properties. Preferably, the adhesive is a medical grade, non-irritating, hydrogel of any suitable type known to those skilled in the art. Hydrogel adhesives are well known in the art, examples of such adhesives are described in U.S. Pat. No. 4,768,523 and U.S. Pat. No. 4,226,232, both of which are hereby incorporated by reference in their entirety. A hypoallergenic acrylic pressure sensitive adhesive may also be employed.

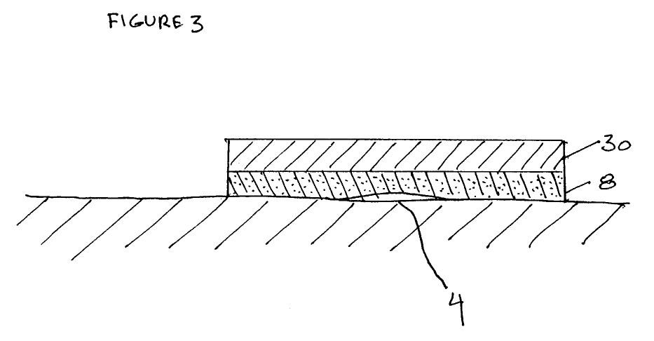

Referring to FIG. 3, the matrix layer 8 is in direct contact with the affected skin area 4. Protecting the matrix is the backing layer 30.

The polymer matrix also includes a skin conditioner and penetration enhancer which may be methyl sulfonyl methane, glucosamine hydrochloride, glucosamine sulfate, N-acetyl glucosamine and chondriotin sulfate, Vitamin E, biotin, methionine, coal tar, aloe vera, allantoin, collagen, with hazel, sodium hyaluronate, boswellin, curcumin, ginger, aswagandha, diphenhydramine hydrochloride, glycyrrhizinic acid, glycol salicylate, phellodendron extract, zinc and copper, either alone or in mixtures of two or more. In its preferred configuration, the skin conditioner and penetration enhancer are methyl sulfonyl methane, glucosamine and chondroitin or combinations thereof.

Optionally, the polymer matrix includes a drug ingredient. These may include yucca or yucca extract, vitamins, amino acids, lidocaine, tetracaine, benzocaine, ketoprofin, indomethacin, capsaicin, methyl salicylate, thymol, tocopherol acetate, menthol, camphor, eucalyptus and menthol esters either alone or in combinations of two or more.

Additionally, the polymer matrix may have dispersed within it one or more antimicrobial agents including isopropyl alcohol, povidone iodine, mercurochrome, hydrogen peroxide, benzoyl peroxide, retinoic acid, tetracycline, chlorohexidine gluconate, erythromycin, miconazole, acyclovir, isotretinoin, hexachlorophene, silver nitrate, acetic acid, salicyclic acid and the like.

The polymer matrix is often gelled by physical means such as using colloidal material, chemical or curing though UV light, irradiation or ethylene oxide exposure.

The method of delivering agents to the skin using the carrier patch involves obtaining the patch, removing the protective sheet to expose the polymer matrix to the skin, and placing the patch on the skin. The polymer matrix adheres to the skin through its adhesive properties. Subsequently, the patch is removed from the skin after the expiration of a specified time period, such as one day. One preferred protocol is to replace with patch twice per day.

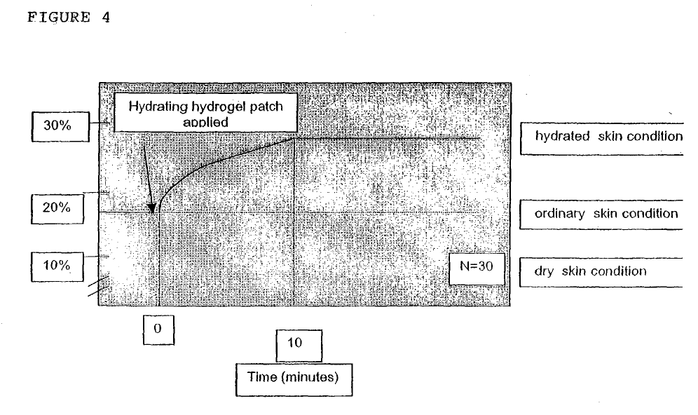

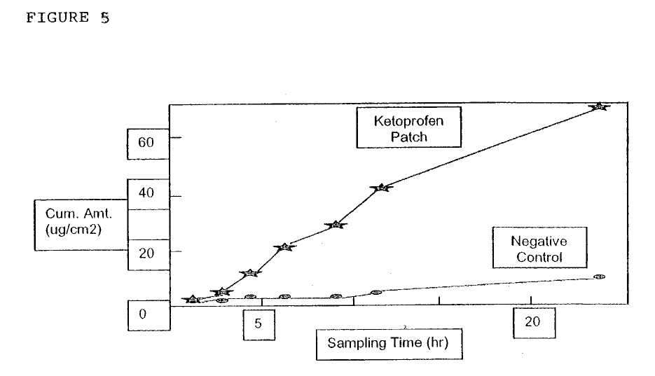

The utility of the present invention is illustrated by FIGS. 4 and 5. Referring to FIG. 4, a hydrating hydrogel patch increases the water concentration in keratin over a period of time creating a hydrated skin condition. FIG. 5 demonstrates that optimal skin permeation of ketoprofen occurs after a relatively long treatment period of over 20 hours utilizing a ketoprofen patch. Specifically, skin permeation of 60 ug/cm2 occurs after approximately 20 hours of contact with a ketoprofen patch.

Additionally, the patch may either be non-sterile, or sterilized if desired. The patch may be used on both humans and animals.

What is claimed is:

1. An adhesive patch comprising:
a backing;
a polymer matrix applied to said backing; and
a topically applied composition contained within said polymer matrix.

2. The adhesive patch of claim 1 in which said backing is a non-woven substrate manufactured from a natural material, naturally derived material, or a synthetic material or a mixture thereof.

3. The adhesive patch of claim 2 in which said natural material is selected from the group consisting of the cotton, flax, linen, paper or cellulose families.

4. The adhesive patch of claim 2 in which said naturally derived material is selected from the group consisting of viscose rayon or solvent spun rayon.

5. The adhesive patch of claim 2 in which said synthetic material is selected from the group consisting of the polyamide, polyester, polyolefin, polyurethane or polyacrylate families.

6. The adhesive patch of claim 1 in which said backing is a non-occlusive film selected from the group consisting of polypropylene, polyurethane and polyethylene.

7. The adhesive patch of claim 1 in which said polymer matrix is a hydrogel matrix.

8. The adhesive patch of claim 1 in which said polymer matrix is an oil based, latex gel.

9. The adhesive patch of claim 1 in which said topically applied composition contains a skin conditioner.

10. The adhesive patch of claim 9 in which said skin conditioner is selected from the group consisting of methyl sulfonyl methane, glucosamine, vitamin E, biotin, methionine, coal tar, aloe vera, allantoin, collagen, witch hazel, sodium hyaluronate, boswellin, curcumin, ginger, aswagandha, diphenhydramine hydrochloride, glycyrrhizinic acid, glycol salicylate, phellodenron extract, zinc or copper.

11. The adhesive patch of claim 1 in which said topically applied composition further comprises a pharmaceutical compound selected from the group consisting of yucca, yucca extract, vitamins, amino acids, lidocaine, tetracaine, benzocaine, ketoprofen, indomethacin, capasaicin, methyl salicylate, thymol, tocopherolacetate, menthol, camphor, eucalyptus, and menthol esters.

12. The adhesive patch of claim 1 in which said polymer matrix includes purified water, glycerin, polyacylate, sorbitol, kaolin, CMC (carboxylmethyl cellulose), alcohol, castor oil, TWEEN 80 (polyoxyethylene sorbitan monooleate) and citric acid.

13. The adhesive patch of claim 1, in which said polymer matrix is removably covered by a protective layer.

14. An adhesive patch carrying a topically applied composition comprising:
a backing;
a hydrogel matrix applied to said backing, said hydrogel matrix containing purified water, glycerin, polyacylate, sorbitol, kaolin, CMC (carboxylmethyl cellulose), alcohol, castor oil, TWEEN 80 (polyoxyethylene sorbitan monooleate) and citric acid;
a topically applied composition contained within said hydrogel matrix, said topically applied composition includes a skin conditioner selected from the group consisting of methyl sulfonyl methane, glucosamine and chondroitin; a protective layer removably covering said polymer matrix.

15. The adhesive patch of claim 14 in which said backing is a non-woven substrate manufactured from a natural material, naturally derived material, or synthetic material, or a mixture thereof.

16. The adhesive patch of claim 15 in which said natural material is selected from the group consisting of the cotton, flax, linen, paper or cellulose families.

17. The adhesive patch of claim 15 in which said naturally derived material is selected from the group consisting of viscose rayon or solvent spun rayon.

18. The adhesive patch of claim 17 in which said synthetic material is selected from the group consisting of the polyamide, polyester, polyolefin, polyurethane and polyacrylate families.

19. The adhesive patch of claim 14 in which said backing is a non-occlusive film selected from the group consisting of polypropylene, polyurethane and polyethylene.

20. The adhesive patch of claim 14 in which said topically applied compound includes a pharmacuetical compound selected from the group consisting of yucca, yucca extract, vitamins, amino acids, lidocaine, tetracaine, benzocaine, ketoprofen, indomethacin, capasaicin, methyl salicylate, thymol, tocopherolacetate, menthol, camphor, eucalyptus and menthol esters.
